(19) United States
(12) Patent Application Publication
(10) Pub. No.: US 2006/0217734 A1
(43) Pub. Date: Sep. 28, 2006

(54) Femoral resection guide apparatus and method

(75) Inventors: Adam H. Sanford, Warsaw, IN (US); Scott J. Steffensmeier, Warsaw, IN (US)

Correspondence Address: ZIMMER TECHNOLOGY - ROBERTS, P.O. BOX 1268, ALEDO, TX, 76008, US

(73) Assignee: Zimmer Technology, Inc.

(21) Appl. No.: 11/075,562

(22) Filed: Mar. 9, 2005

(51) Int. Cl. A61B 17/58

(52) U.S. Cl. 606/88

(57) ABSTRACT

An apparatus for resecting a distal femoral condyle includes a resection guide defining a first bone fixation aperture extending about an axis. The resection guide further defines a first bone saw slot and a second bone saw slot. The resection guide is configured to position the first bone fixation aperture relative to the condyle. The resection guide is further configured to concurrently arcuately translate the first bone saw slot and the second bone saw slot relative to the axis. A knee replacement kit includes a femoral implant defining a first plan contour. The kit further includes a resection guide defining a second plan contour modeling the first plan contour.

### FIELD OF THE INVENTION

The present invention relates generally to the field of orthopaedics, and, more particularly, to an apparatus and method for resecting a distal femoral condyle.

### BACKGROUND

When one side of a person's knee has deteriorated but the other side remains relatively healthy, a partial knee replacement may be desirable. In a partial knee replacement, the deteriorated side of the joint is replaced with a prosthesis but the healthy side is spared. Like many arthroplastic procedures, a partial knee replacement typically includes using special saws and/or other tools to resect the affected bones into suitable geometries with suitable clearances for receiving their respective prosthetic components. After a partial knee replacement, complications may result if either of the affected bones (i.e., the proximal tibia and the distal femur) was not resected properly. Such complications can include accelerated wear of the prosthesis; cracking or fracture of the affected and/or the healthy parts of the proximal tibia and distal femur; loosening, excessive rotation or loss of motion of the prosthesis; and/or angular deformity of the joint.

A resection guide is a jig or template configured to facilitate a desired cutting angle for saw blades or other resection tools. Conventional resection guides are used somewhat similarly to the manner in which a carpenter uses a miter box to achieve a desired angle for cutting wood. Notwithstanding substantial advantages provided by resection guides, surgeons still must typically determine final resection locations and orientations based in large part on experience and with understandings that prosthetic components are available only in a limited number of sizes. In a partial knee replacement, the surgeon typically makes the tibial cut as close to the proximal end of the affected tibia as practical, considering the amount of the proximal tibia that must be removed due to the deterioration, plus whatever additional clearance spacing is required to accommodate the closest standard sized tibial prosthetic component. After making initial tibial and femoral cuts, the surgeon may assemble and apply a provisional (i.e., trial) prosthesis to the joint and analyzes the results. To adjust the fit and biomechanics of the prosthesis, the surgeon can replace the tibial component with one of different thickness and/or remove more bone. Typically, the surgeon repeats such trial and error procedures until achieving a desired limb alignment and soft tissue balance for the prosthesis. Even with some conventional resection guides, this approach can be undesirably time consuming and at times the surgeon can remove more bone than necessary. Additionally, minimally invasive surgical techniques are becoming increasingly popular. Minimally invasive surgeries employ, among other things, considerably smaller incisions and tighter working spaces than historical techniques in efforts to reduce patient trauma and accelerate recoveries.

Thus, there is a need for a resection guide that reduces the trial and error required for a partial knee replacement. Further, there is a need for a small resection guide that is suitable for use in minimally invasive surgical procedures.

### SUMMARY OF THE INVENTION

The present invention provides an apparatus for resecting a distal femoral condyle. The apparatus includes a resection guide defining a first bone fixation aperture extending about an axis. The resection guide further defines a first bone saw slot and a second bone saw slot. The resection guide is configured to position the first bone fixation aperture relative to the condyle. The resection guide is further configured to concurrently arcuately translate the first bone saw slot and the second bone saw slot relative to the axis.

The present invention provides an apparatus for resecting a distal femoral condyle. The apparatus includes means for defining an axis, means, coupled to the axis defining means, for defining a first bone saw slot, means, coupled to the axis defining means, for defining a second bone saw slot, means, coupled to the axis defining means, for positioning the axis relative to the condyle, and means, coupled to the axis defining means, for concurrently arcuately translating the first bone saw slot and the second bone saw slot relative to the axis.

The present invention provides a knee replacement kit including a femoral implant defining a first plan contour. The kit further includes a resection guide defining a second plan contour modeling the first plan contour.

The present invention provides a kit for resecting a distal femoral condyle. The kit includes a femoral implant defining a first plan contour. The kit further includes a resection guide defining a second plan contour modeling the first plan contour. The resection guide further defines a first bone fixation aperture extending about an axis. The resection guide further defines a first bone saw slot and a second bone saw slot. The resection guide is configured to position the first bone fixation aperture based on the anterior-posterior dimension of the distal femur. The resection guide is further configured to concurrently arcuately translate the first bone saw slot and the second bone saw slot relative to the axis.

The present invention provides a method for resecting a distal femoral condyle. The method includes the steps of defining an axis, defining a first cutting path, defining a second cutting path, positioning the axis relative to the condyle, concurrently arcuately translating the first cutting path and the second cutting path relative to the axis, resecting the distal femur along the first cutting path, and resecting the distal femur along the second cutting path.

The above-noted features and advantages of the present invention, as well as additional features and advantages, will be readily apparent to those skilled in the art upon reference to the following detailed description and the accompanying drawings, which include a disclosure of the best mode of making and using the invention presently contemplated.

### BRIEF DESCRIPTION OF THE DRAWINGS

FIG. 1 shows a perspective view of an exemplary unicondylar femoral implant according to the present invention positioned on a distal femur, with the distal femur in 90 degrees flexion relative to a proximal tibia of an intra-operative left knee;

FIG. 2 shows an anterior plan view of the exemplary implant of FIG. 1 positioned on the distal femur, with the distal femur in 90 degrees flexion relative to the proximal tibia;

FIG. 3 shows a perspective view of an exemplary resection guide apparatus according to the present invention positioned the distal femur (generally in place of the exemplary implant of FIG. 1), with the distal femur in 90 degrees flexion relative to the proximal tibia;

FIG. 4 shows an anterior plan view of the exemplary apparatus of FIG. 3 positioned on the distal femur, with the distal femur in 90 degrees flexion relative to the proximal tibia;

FIG. 5 shows a cross-sectional view (along line 5-5 FIG. 4) of the exemplary apparatus of FIG. 3 attached to the distal femur by exemplary bone fasteners; and

FIG. 6 shows cross-section (taken along line 6A-6A of FIG. 2) of the exemplary implant of FIG. 1 superimposed on a cross-section (taken along line 6B-6B of FIG. 4) of the exemplary apparatus of FIG. 3;

FIG. 7 shows the cross-section of the exemplary implant (alone); and

FIG. 8 shows the cross-section of the exemplary apparatus (alone).

### DETAILED DESCRIPTION OF EXEMPLARY EMBODIMENT(S)

Like reference numerals refer to like parts throughout the following description and the accompanying drawings. As used herein, the terms “medial,” “medially,” and the like mean pertaining to the middle, in or toward the middle, and/or nearer to the middle of the body when standing upright. Conversely, the terms “lateral,” “laterally,” and the like are used herein as opposed to medial. For example, the medial side of the knee is the side closest to the other knee and the closest sides of the knees are medially facing, whereas the lateral side of the knee is the outside of the knee and is laterally facing. Further, as used herein the term “superior” means closer to the top of the head and/or farther from the bottom of the feet when standing upright. Conversely, the term “inferior” is used herein as opposed to superior. For example, the heart is superior to the stomach and the superior surface of the tongue rests against the palate, whereas the stomach is inferior to the heart and the palate faces inferiorly toward the tongue. Also, as used herein the terms “anterior,” “anteriorly,” and the like mean nearer the front or facing away from the front of the body when standing upright, as opposed to “posterior,” “posteriorly,” and the like, which mean nearer the back or facing away from the back of the body. Additionally, as used herein the term “unicondylar” and inflections thereof mean configured to fit onto and/or replace a single one of either a medial condyle or a corresponding lateral condyle of a joint. Nevertheless, it is noted that the particular directional and/or positional terms and inflections thereof used herein are merely for clarity of exposition, and at times they may be somewhat arbitrary or interchangeable as known in the art. For example, although the present invention is described herein relative to exemplary left knee medial condyle replacements, it should be appreciated that in many cases corresponding lateral condylar embodiments and/or corresponding right knee embodiments may be made by simply exchanging “medial” and “lateral” features where appropriate (i.e., mirroring) as known in the art.

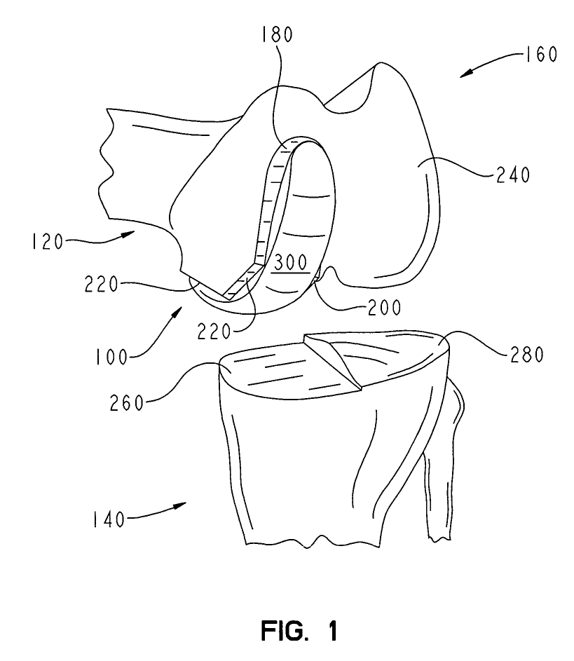

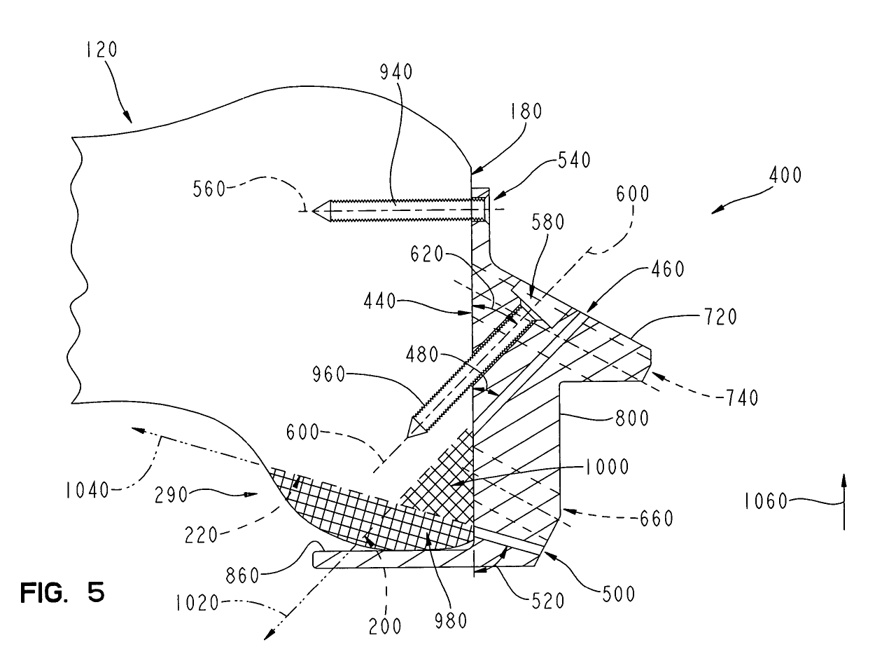

FIG. 1 shows a perspective view of an exemplary unicondylar femoral implant 100 according to the present invention positioned on a distal femur 120, with distal femur 120 in 90 degrees flexion relative to a proximal tibia 140 of an intra-operative left knee 160. In FIG. 1, distal femur 120 includes a substantially planar distal femoral resection surface 180, a substantially planar chamfer resection surface 200, and a substantially planar posterior resection surface 220 for accommodating implant 100, and further includes a natural lateral distal femoral condyle 240. Proximal tibia 140 includes a substantially planar resected surface 260 for accommodating typical corresponding prosthetic tibial components (not shown), and further includes a natural lateral tibial plateau 280. Among other things, implant 100 is configured to replace a distal femoral condyle 290 (see FIG. 5) in a partial knee replacement. In the exemplary embodiment, implant 100 is made from a cobalt chrome alloy. In alternative embodiments, implant 100 may be made from any other suitable biocompatible material(s). As discernable in FIG. 1, implant 100 includes a generally convex outwardly facing surface 300.

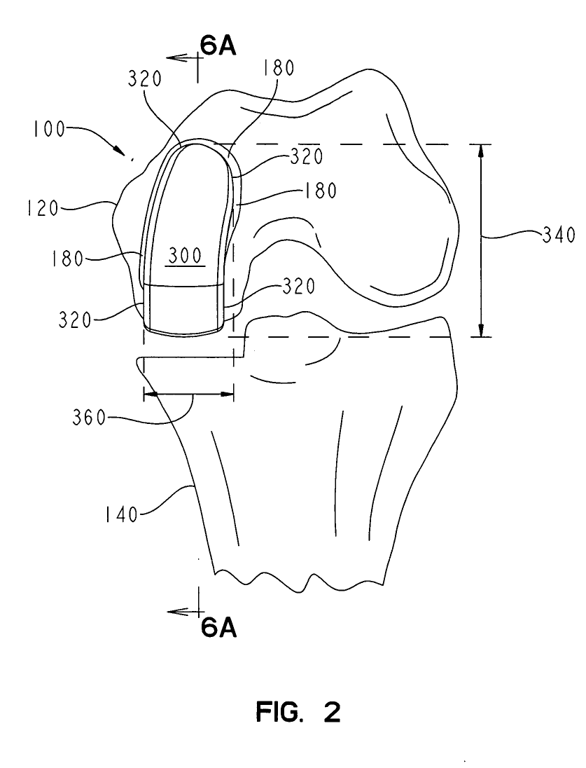

FIG. 2 shows an anterior plan view of implant 100 positioned on distal femur 120, with distal femur 120 in 90 degrees flexion relative to proximal tibia 140. As discernable in FIG. 2, implant 100 defines a plan outline or plan contour 320 having an anterior-posterior span 340 and a medial-lateral span 360. At first glance span 340 might be viewed as a superior-inferior dimension. However, it should be remembered that in FIG. 2 distal femur 120 is shown in 90 degrees flexion. Accordingly, span 340 is actually an anterior-posterior dimension when distal femur 120 is thought of in the conventional upright standing position (i.e., 0 degrees of flexion). Surface 300, among other things, is also at least partially discernable in FIG. 2.

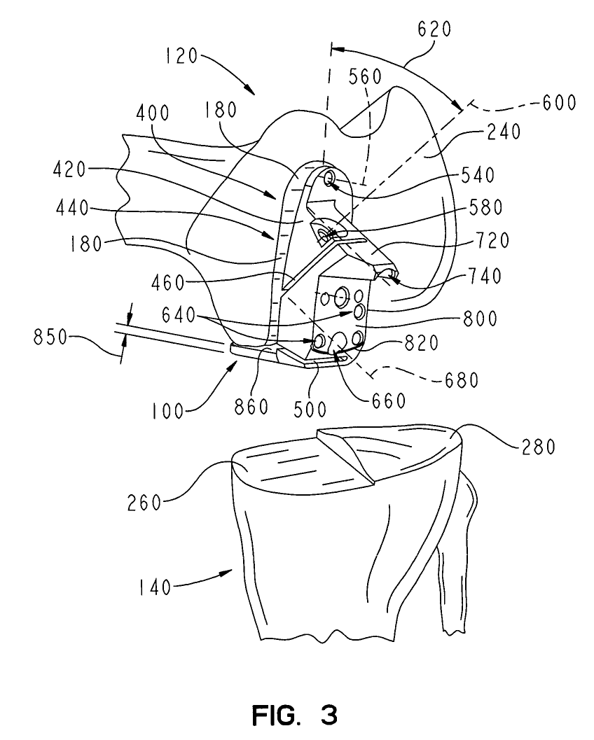

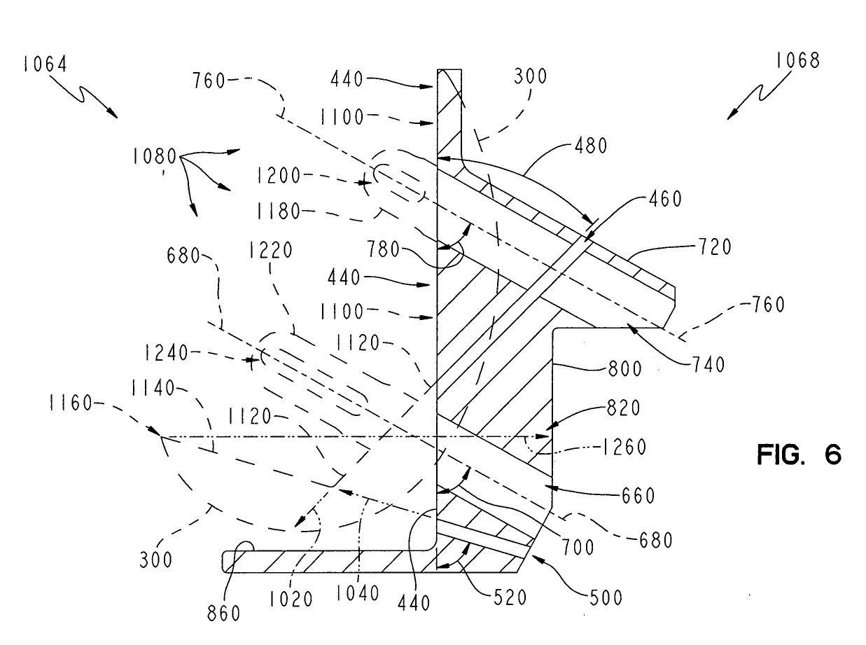

FIG. 3 shows a perspective view of an exemplary resection guide apparatus 400 according to the present invention positioned on distal femur 120 (generally in place of implant 100; see FIG. 1 and FIG. 2), with distal femur 120 in 90 degrees flexion relative to proximal tibia 140. In FIG. 3, distal femur 120 is shown including surface 180 to facilitate positioning of apparatus 400. However, it is noted that in FIG. 3 distal femur 120 is shown prior to additional resections according to the present invention that substantially remove condyle 290 (see FIG. 5) to provide surface 200 (see FIG. 1 and FIG. 5) and surface 220 (see FIG. 1 and FIG. 5). Among other things, apparatus 400 is configured to provide desired cutting angles for saw blades or other resection tools (not shown). In the exemplary embodiment, apparatus 400 is made from stainless steel. In alternative embodiments, apparatus 100 may be made from any other suitable surgical grade material(s). Apparatus 400 includes a body portion 420. Portion 420 includes a substantially planar surface 440 (see also FIG. 5 and FIG. 6), and defines a generally planar bone saw slot 460 extending through portion 420 at an angle 480 (see FIG. 5 and FIG. 6) relative to surface 440. In the exemplary embodiment, angle 480 is about 45 degrees. Portion 420 also defines a generally planar bone saw slot 500 extending through portion 420 at an angle 520 (see FIG. 5 and FIG. 6) relative to surface 440. In the exemplary embodiment, angle 520 is about 65 degrees. Portion 420 also defines a generally cylindrical bone fixation aperture 540 (see also FIG. 5) extending through portion 420 about an axis 560 (see also FIG. 5) that is perpendicular to surface 440. Portion 420 also defines a generally cylindrical bone fixation aperture 580 (see also FIG. 5) extending through portion 420 about an axis 600 (see also FIG. 5). Axis 600 is angularly disposed from surface 440 by an angle 620 (see also FIG. 5). In the exemplary embodiment, angle 620 is about 45 degrees. Portion 420 also defines a plurality of additional generally cylindrical bone fixation apertures 640 extending through portion 420 perpendicularly to surface 440. Portion 420 also defines a generally cylindrical drill bit aperture 660 (see also FIG. 6) extending through portion 420 about an axis 680 (see also FIG. 6). Axis 680 is angularly disposed from surface 440 by an angle 700 (see FIG. 6). In the exemplary embodiment, angle 700 is about 60 degrees. Apparatus 400 further includes a flange 720 protruding from portion 420. Portion 420 and flange 720 together define a generally cylindrical drill bit aperture 740 (see also FIG. 6) extending through portion 420 and flange 720 about an axis 760 (see FIG. 6). Axis 760 is angularly disposed from surface 440 by an angle 780 (see FIG. 6). In the exemplary embodiment, angle 780 is about 60 degrees. Portion 420 also includes a generally outwardly facing surface 800 (see also FIG. 5 and FIG. 6) having an etched or engraved line 820 proximal to slot 500 and arcing generally medially-laterally across surface 800. Apparatus 400 further includes a tab 840 extending from portion 420. Tab 840 has a thickness 850, and includes a substantially planar surface 860 that is roughly perpendicular to surface 440. Condyle 240 and proximal tibia 140 (including surface 260 and plateau 280), among other things, are also at least partially discernable in FIG. 3.

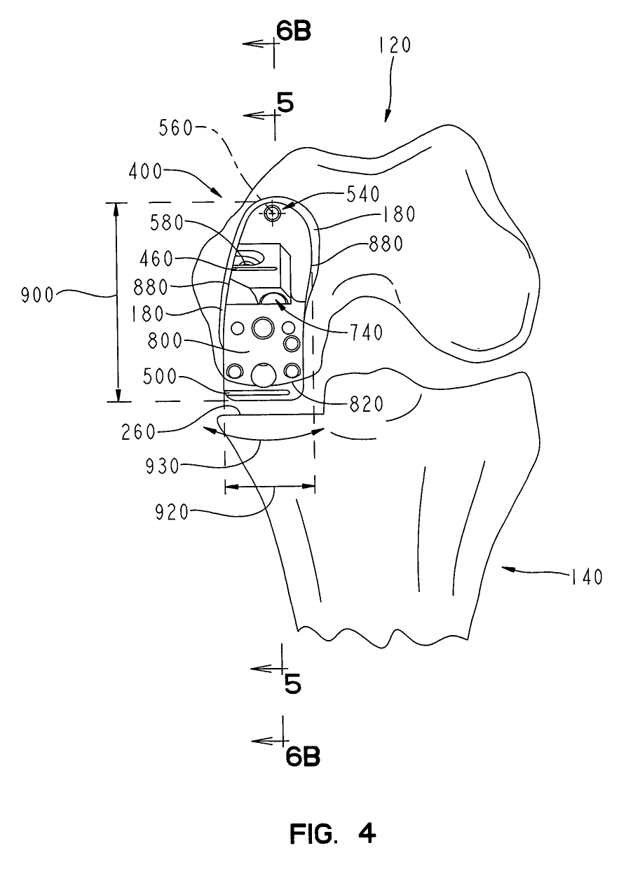

FIG. 4 shows an anterior plan view of apparatus 400 positioned on distal femur 120, with distal femur 120 in 90 degrees flexion relative to proximal tibia 140. It is noted that in FIG. 4 distal femur 120 is shown prior to resections according to the present invention that substantially remove condyle 290 (see FIG. 5) to provide surface 200 (see FIG. 1 and FIG. 5) and surface 220 (see FIG. 1 and FIG. 5). As discernable in FIG. 4, apparatus 400 defines a plan outline or plan contour 880. Contour 880 models contour 320 (compare FIG. 4 and FIG. 2). Contour 880 has, among other things, an anterior-posterior span 900 that models span 340 (see FIG. 2). Span 900 is roughly equivalent to span 340 (see FIG. 2) plus thickness 850 (see FIG. 3). Contour 880 also has, among other things, a medial-lateral span 920 that models span 360 (see FIG. 2). Span 920 is roughly equivalent to span 360. At first glance span 900 might be viewed as a superior-inferior dimension. However, it should be remembered that in FIG. 4 distal femur 120 is shown in 90 degrees flexion. Accordingly, span 900 is actually an anterior-posterior dimension when distal femur 120 is thought of in the conventional upright standing position (i.e., 0 degrees of flexion). As further discernable in FIG. 4, apparatus 400 is also geometrically configured to concurrently arcuately translate slot 460 and slot 500 relative to axis 560 (see also FIG. 3 and FIG. 5) as indicated generally by directional lines 930. Slot 460, slot 500, aperture 540, aperture 580, aperture 660, aperture 740, surface 800, and line 820, among other things, are also at least partially discernable in FIG. 4.

FIG. 5 shows a cross-sectional view (along line 5-5 FIG. 4) of apparatus 400 attached to distal femur 120 by an exemplary bone fastener 940 and an exemplary bone fastener 960. In the exemplary embodiment, fastener 940 and fastener 960 are implemented as conventional bone screws. In alternative embodiments, fastener 940 and/or fastener 960 may be nails, pins, or any other suitable fastener(s). In FIG. 5, distal femur 120 is shown including surface 180, which facilitates positioning of apparatus 400. However, it is noted that in FIG. 5 distal femur 120 is shown prior to additional resections according to the present invention that remove a portion 980 and a portion 1000 from condyle 290 to provide surface 220 (see also FIG. 1) and surface 200 (see also FIG. 1), respectively. As discernable in FIG. 5, slot 460 defines a cutting path 1020 for a bone saw or other suitable resection tool (not shown), and slot 500 defines another cutting path 1040 for the bone saw or other suitable resection tool (not shown). Among other things, slot 460, slot 500, aperture 540, aperture 580, fastener 940, and fastener 960 are positioned and configured to preclude fastener 940 and/or fastener 960 from crossing or otherwise obstructing path 1020 and/or path 1040. As further discernable in FIG. 5, apparatus 400 is also geometrically configured to position aperture 540 (and thus, axis 560) relative to surface 860 (and thus, relative to condyle 290 when surface 860 abuts condyle 290). Angle 480, angle 520, axis 560, axis 600, angle 620, aperture 660, flange 720, aperture 740, surface 800, and surface 860, among other things, are also at least partially discernable in FIG. 5, while apertures 640 and line 820 are omitted from FIG. 5 for clarity. FIG. 5 also shows a directional line 1060.

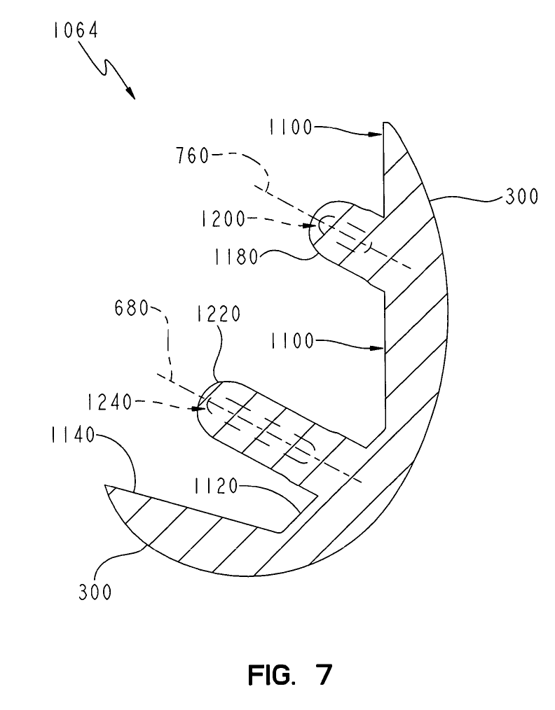

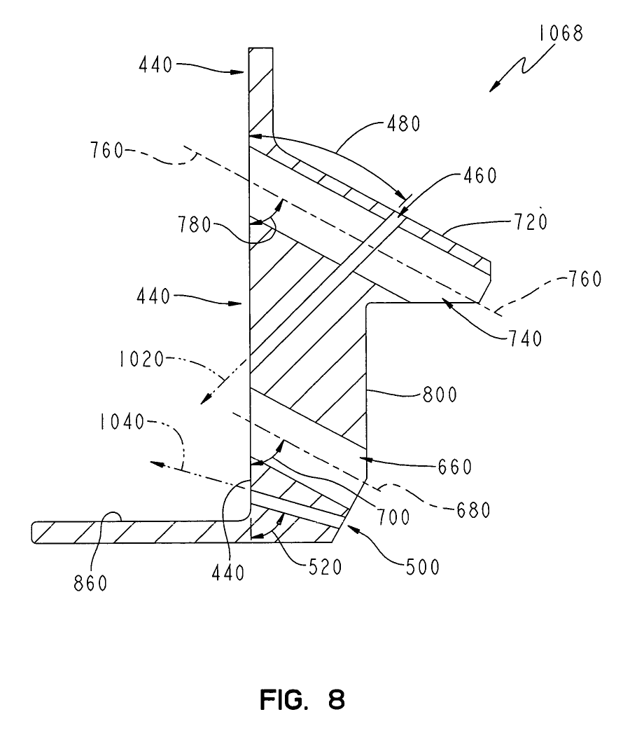

FIG. 6 shows a cross-section 1064 (taken along line 6A-6A of FIG. 2) of implant 100 (see FIG. 1 and FIG. 2) superimposed on a cross-section 1068 (taken along line 6B-6B of FIG. 4) of apparatus 400 (see FIG. 3 and FIG. 4); FIG. 7 shows cross-section 1064 (alone); and FIG. 8 shows cross-section 1068 (alone). Implant 100 includes a generally concave, facetted (i.e., piecewise substantially planar) inwardly facing surface 1080. Surface 1080 includes a substantially planar facet 1100 (shown in coincidence with surface 440 of apparatus 400), further includes a substantially planar facet 1 120 extending angularly from facet 1100, and further includes a substantially planar facet 1140 extending angularly from facet 1120. Facet 1140 includes a posterior edge 1160. Implant 100 also includes an axial peg or post 1180 extending away from facet 1100. Post 1180 defines an elongated sidewall groove 1200. Implant 100 also includes an axial peg or post 1220 extending away from facet 1120. Post 1220 defines an elongated sidewall groove 1240. Implant 100 and apparatus 400 are configured such that when cross-section 1064 is superimposed on cross-section 1068, facet 1100 coincides with surface 440, facet 1120 coincides with cutting path 1020, facet 1140 coincides with cutting path 1040, post 1180 is axially aligned along axis 760, post 1120 is axially aligned along axis 680, and surface 300 tangentially coincides with surface 860. Further, line 820 models edge 1160 by coinciding with a projection 1260 (through apparatus 400 to surface 800) of edge 1160. Surface 440, slot 460, angle 480, slot 500, angle 520, aperture 660, axis 680, angle 700, flange 720, aperture 740, axis 760, and angle 780, among other things, are also at least partially discernable in FIG. 6, while aperture 540 and aperture 580 are omitted from FIG. 6 for clarity of depiction.

In preparation for using apparatus 400, a surgeon or other user suitably resects distal femur 120 and proximal tibia 140 to provide surface 180 and surface 260, respectively (see FIG. 1) in a known manner. Then, in using apparatus 400, the user moves surface 440 of apparatus 400 along surface 180 of distal femur 120 (as indicated generally by directional line 1060; see FIG. 5) until surface 860 of apparatus 400 contacts portion 980 of condyle 290 (see FIG. 5). As the user moves surface 440 along surface 180, apparatus 400 positions aperture 540 (and thus, axis 560) relative to surface 860 (and thus, relative to condyle 290).

Next, the user rotationally repositions apparatus 400 about axis 560 (as indicated generally by directional lines 930) until apparatus 400 arcuately translates slot 500 (relative to axis 560) into parallelism with surface 260 of proximal tibia 140 (see FIG. 4). As contour 880 of apparatus 400 models contour 320 of implant 100 (see FIG. 2 and FIG. 4) and line 820 of apparatus 400 (see FIG. 4 and FIG. 6) models edge 1160 of implant 100 (see FIG. 6), the user may preliminarily assess the size and ultimate orientation of implant 100 (without necessarily removing more bone or constructing more cumbersome provisional assemblies) by referring to contour 880 and line 820. As desired, the user may try out alternative embodiments of apparatus 400 (each modeling corresponding respective alternative embodiments of implant 100) over a range of various contours and/or sizes until deciding on a most suitable embodiment.

Next, the user inserts fastener 940 through aperture 540 and advances fastener 940 into distal femur 120 tightly enough to hold surface 440 against surface 180, but not so tightly as to prevent rotation of apparatus 400 about axis 560. The user rotationally repositions apparatus 400 about axis 560 (as indicated generally by directional lines 930) until apparatus 400 arcuately translates slot 500 (relative to axis 560) into parallelism with surface 260 of proximal tibia 140 (see FIG. 4). As apparatus 400 arcuately translates slot 500 relative to axis 560, apparatus 400 also concurrently arcuately translates slot 460 relative to axis 560.

Next, the user inserts fastener 960 through aperture 580 and advances fastener 960 into distal femur 120 to securely attach apparatus 400 to distal femur 120 (see FIG. 5). After installing fastener 960, the user may further advance/tighten fastener 940 as desired. If desired, the user may install additional fasteners (not shown) though apertures 640. Slot 460, slot 500, aperture 540, aperture 580, fastener 940, and fastener 960 cooperate to preclude fastener 940 and/or fastener 960 from crossing or otherwise obstructing path 1020 and/or path 1040. Similarly, slot 460, slot 500, apertures 640, and any additional fasteners cooperate to preclude obstruction of path 1020 and/or path 1040.

After securing apparatus 400 to distal femur 120, the user uses aperture 660 and then aperture 740 for guiding a drill (not shown) to suitably bore into distal femur 120 along axis 680 (see FIG. 6) and axis 760 (see FIG. 6), respectively. The user withdraws the drill and then inserts a suitable bone saw or other resection tool (not shown) through slot 500. With slot 500 guiding the resection tool along cutting path 1040, the user resects portion 980 from condyle 290 (see FIG. 5), which provides surface 220 (see FIG. 1 and FIG. 5). Next, the user removes the resection tool from slot 500 and inserts it into slot 460. With slot 460 guiding the resection tool along cutting path 1020, the user resects portion 1000 from condyle 290 (see FIG. 5), which provides surface 200 (see FIG. 1 and FIG. 5).

After resecting and boring distal femur 120 as discussed above, the user removes apparatus 400 and suitably attaches implant 100 to distal femur 120 such that facet 1100 of implant 100 (see FIG. 6) abuts surface 180 of distal femur 120 (see FIG. 1), facet 1120 of implant 100 (see FIG. 6) abuts surface 200 of distal femur 120 (see FIG. 1), facet 1140 of implant 100 (see FIG. 6) abuts surface 220 of distal femur 120 (see FIG. 1), post 1180 of implant 100 (see FIG. 6) extends into distal femur 120, and such that post 1220 of implant 100 (see FIG. 6) extends into distal femur 120. The user fixes corresponding tibial components (not shown) to proximal tibia 140 as known. In operation, implant 100 (see FIG. 1, FIG. 2, and FIG. 6) emulates portion 980 and portion 1000 of condyle 290 (see FIG. 5) as known.

The foregoing description of the invention is illustrative only, and is not intended to limit the scope of the invention to the precise terms set forth. Further, although the invention has been described in detail with reference to certain illustrative embodiments, variations and modifications exist within the scope and spirit of the invention as described and defined in the following claims.

What is claimed is:

1. An apparatus for resecting a distal femoral condyle, the apparatus comprising:
a resection guide defining a first bone fixation aperture extending about an axis, the resection guide further defining a first bone saw slot and a second bone saw slot;
wherein the resection guide is configured to position the first bone fixation aperture relative to the condyle, and the resection guide is further configured to concurrently arcuately translate the first bone saw slot and the second bone saw slot relative to the axis.

2. The apparatus of claim 1, wherein the resection guide includes a body having a first substantially planar surface and a tab having a second substantially planar surface, the first bone saw slot extends at an angle of about 45 degrees relative to the first substantially planar surface, the second bone saw slot extends at an angle of about 65 degrees relative to the first substantially planar surface, and the second substantially planar surface extends at an angle of about 90 degrees relative to the first substantially planar surface.

3. An apparatus for resecting a distal femoral condyle, the apparatus comprising:
means for defining an axis;
means, coupled to the axis defining means, for defining a first bone saw slot;
means, coupled to the axis defining means, for defining a second bone saw slot;
means, coupled to the axis defining means, for positioning the axis relative to the condyle; and
means, coupled to the axis defining means, for concurrently arcuately translating the first bone saw slot and the second bone saw slot relative to the axis.

4. The apparatus of claim 3, further comprising:
means, coupled to the translating means, for concurrently fixing a first position of the first bone saw slot and a second position of the second bone saw slot.

5. The apparatus of claim 4, wherein the fixing means precludes obstruction of the first bone saw slot and the second bone saw slot.

6. A knee replacement kit, comprising:
a femoral implant defining a first plan contour; and
a resection guide defining a second plan contour modeling the first plan contour.

7. The kit of claim 6, wherein the resection guide is unicondylar.

8. The kit of claim 7, wherein the femoral implant is unicondylar.

9. The kit of claim 8, wherein the first plan contour defines a medial-lateral span and the second plan contour models at least the medial-lateral span.

10. The kit of claim 9, wherein the first plan contour defines an anterior-posterior span and the second plan contour models at least the anterior-posterior span.

11. A kit for resecting a distal femoral condyle, the kit comprising:
a femoral implant defining a first plan contour; and
a resection guide defining a second plan contour modeling the first plan contour, the resection guide further defining a first bone fixation aperture extending about an axis, and the resection guide further defining a first bone saw slot and a second bone saw slot;
wherein the resection guide is configured to position the first bone fixation aperture based on the anterior-posterior dimension of the distal femur, and the resection guide is further configured to concurrently arcuately translate the first bone saw slot and the second bone saw slot relative to the axis.

12. The kit of claim 11, wherein the resection guide is unicondylar.

13. The kit of claim 12, wherein the femoral implant is unicondylar.

14. The kit of claim 13, wherein the first plan contour defines a medial-lateral span and the second plan contour models at least the medial-lateral span.

15. The kit of claim 14, wherein the first plan contour defines an anterior-posterior span and the second plan contour models at least the anterior-posterior span.

16. The kit of claim 15, wherein the resection guide includes a body having a first substantially planar surface and a tab having a second substantially planar surface, the first bone saw slot extends at an angle of about 45 degrees relative to the first substantially planar surface, the second bone saw slot extends at an angle of about 65 degrees relative to the first substantially planar surface, and the second substantially planar surface extends at an angle of about 90 degrees relative to the first substantially planar surface.

17. A method for resecting a distal femoral condyle, the method comprising the steps of:
defining an axis;
defining a first cutting path;
defining a second cutting path;
positioning the axis relative to the condyle;
concurrently arcuately translating the first cutting path and the second cutting path relative to the axis;
resecting the distal femur along the first cutting path; and
resecting the distal femur along the second cutting path.

18. The method claim 17, further comprising the step of modeling a plan contour of a femoral implant.

19. The method of claim 18, wherein the modeling step includes modeling a medial-lateral span of the femoral implant.

20. The method of claim 19, wherein the modeling step includes modeling an anterior-posterior span of the femoral implant.

21. The method of claim 20, wherein the modeling step includes modeling a medial-lateral span of the femoral implant concurrently with the modeling of the anterior-posterior span of the femoral implant.

22. The method of claim 21, further comprising the steps of:
concurrently fixing a first position of the first cutting path and a second position of the second cutting path; and
precluding obstruction of the first cutting path and the second cutting path concurrently with the fixing step.

23. The method claim 17, further comprising the step of modeling a plan contour of a unicondylar femoral implant.

24. The method of claim 23, wherein the modeling step includes modeling a medial-lateral span of the unicondylar femoral implant.

25. The method of claim 24, wherein the modeling step includes modeling an anterior-posterior span of the unicondylar femoral implant.

26. The method of claim 25, wherein the modeling step includes modeling a medial-lateral span of the unicondylar femoral implant concurrently with the modeling of the anterior-posterior span of the unicondylar femoral implant.

27. The method of claim 26, further comprising the steps of:
concurrently fixing a first position of the first cutting path and a second position of the second cutting path; and
precluding obstruction of the first cutting path and the second cutting path concurrently with the fixing step.

28. The method of claim 23, wherein the modeling step includes modeling a posterior edge of the unicondylar femoral implant.
